(19) United States
(12) Patent Application Publication
(10) Pub. No.: US 2026/0108731 A1
(43) Pub. Date: Apr. 23, 2026

(54) BEAUTY DEVICE

(71) Applicant: Shenzhen Ulike Smart Electronics Co., Ltd., Shenzhen (CN)

(72) Inventors: WEISONG ZHANG, Shenzhen (CN); FUJIANG LIN, Shenzhen (CN); SHUI YU, Shenzhen (CN); Fei YU, Shenzhen (CN); LIANJIE LIU, Shenzhen (CN)

(21) Appl. No.: 19/145,920

(22) PCT Filed: Aug. 29, 2024
(86) PCT No.: PCT/CN2024/115617

§ 371 Date: Jul. 4, 2025

(30) Foreign Application Priority Data
Jan. 10, 2024 (CN) 202420075251.5
Jan. 22, 2024 (CN) 202420154666.1
Jan. 22, 2024 (CN) 202420154692.4
Apr. 12, 2024 (CN) 202420767983.0

(51) Int. Cl. A61N 1/32; G01R 31/28

(52) U.S. Cl. CPC A61N 1/328; G01R 31/282

(57) ABSTRACT

A beauty device including an isolation circuit and a working circuit is provided. A first terminal of the isolation circuit is configured to access a test voltage or output a driving signal, and a second terminal of the isolation circuit is electrically connected to the working circuit. The working circuit is configured to output the driving signal to the isolation circuit.

### TECHNICAL FIELD

The present disclosure relates to the field of beauty devices, in particular to a beauty device.

### BACKGROUND

To meet safety standards, radio frequency products such as beauty devices must undergo a high-voltage withstand test and can only be shipped after passing the test. However, in the related art, the radio frequency products such as the beauty devices are unable to support a high-voltage test of 1000 Volt.

### SUMMARY

In order to solve the problems in the related art, the present disclosure provides a beauty device, realizing the voltage withstand performance of the beauty device.

The present disclosure provides a beauty device, including:
an isolation circuit, a first terminal of the isolation circuit being configured to access a test voltage or output a driving signal; and
a working circuit, the working circuit being electrically connected to a second terminal of the isolation circuit, and configured to output the driving signal to the isolation circuit.

The present disclosure realizes voltage withstand of the beauty device to the test voltage by providing the isolation circuit, which meets the voltage withstand requirement of the beauty device and enhances the safety of the beauty device. Moreover, the isolation circuit realizes the transmission of the driving signal, ensuring normal operation of the beauty device.

### BRIEF DESCRIPTION OF THE DRAWINGS

FIG. 1 is a schematic module diagram of a beauty device according to an embodiment of the present disclosure.

FIG. 2 is a schematic circuit diagram of a working circuit according to an embodiment of the present disclosure.

FIG. 3 is a schematic circuit diagram of a transformer according to an embodiment of the present disclosure.

FIG. 4 is a schematic circuit diagram of a relay according to an embodiment of the present disclosure.

FIG. 5 is a schematic circuit diagram of an isolation optocoupler according to an embodiment of the present disclosure.

FIG. 6 is a schematic structural diagram of a beauty device according to an embodiment of the present disclosure.

FIG. 7 is a front view of the beauty device in FIG. 6.

FIG. 8 is a schematic diagram showing a heat distribution of an electrode assembly of the beauty device in FIG. 6.

FIG. 9 is a front view of a beauty device according to another embodiment of the present disclosure.

FIG. 10 is a front view of a beauty device according to still another embodiment of the present disclosure.

FIG. 11 is a schematic structural diagram of a light guide component according to an embodiment of the present disclosure.

FIG. 12 is a front view of the light guide component in FIG. 11.

FIG. 13 is a schematic cross section diagram of the light guide component in FIG. 12.

FIG. 14 is a partial exploded diagram of the beauty device according to an embodiment of the present disclosure.

FIG. 15 is a schematic diagram showing installation of a light-emitting component and a light guide component according to an embodiment of the present disclosure.

FIG. 16 is a schematic structural diagram of a light-emitting component according to an embodiment of the present disclosure.

FIG. 17 is a schematic structural diagram of a beauty device according to an embodiment of the present disclosure.

FIG. 18 is a partial exploded diagram of the beauty device in FIG. 17.

FIG. 19 is a schematic structural diagram of an elastic connecting component according to an embodiment of the present disclosure.

FIG. 20 is a schematic diagram showing connection between a charging electrode and an elastic connecting component according to an embodiment of the present disclosure.

FIG. 21 is a front view of the elastic connecting component in FIG. 19.

The following specific embodiments will be described in conjunction with the accompanying drawings for further explanation of the present disclosure.

### DETAILED DESCRIPTION OF THE EMBODIMENTS

The present disclosure will be described more completely with reference to the accompanying drawings. Exemplary embodiments of the present disclosure are shown in the accompanying drawings. However, the present disclosure may be implemented in many different forms and should not be interpreted as limited to the exemplary embodiments disclosed herein. These exemplary embodiments are provided to make the present disclosure thorough and complete, and to adequately convey the scope of the present disclosure to those skilled in the art. Similar reference numerals indicate the same or similar components.

The terms used herein are for the purpose of describing the specific exemplary embodiments and are not intended to limit the scope of the present disclosure. As used herein, unless the context clearly indicates otherwise, the singular forms “one”, “a”, and “the” are intended to include the plural forms as well. Furthermore, when used in the present disclosure, the terms “include” and/or “comprise” and/or “have” refer to the presence of stated features, integers, steps, operations, components, and/or groups, but do not preclude the addition or presence of one or more other features, areas, integers, steps, operations, components, and/or their groups.

Unless otherwise defined, all terms used herein (including technical and scientific terms) have the same meaning as commonly understood by those skilled in the art to which the present disclosure belongs. Furthermore, unless defined herein, terms such as those defined in general dictionaries should be interpreted to have meanings consistent with their use in the related art and the content of the present disclosure, and should not be interpreted to have idealized or overly formal meanings.

The exemplary embodiments will be described with reference to the drawings. It should be noted that the components depicted in the drawings are not necessarily shown to scale; and the same or similar components will be denoted with the same or similar reference numerals or described with similar technical terms.

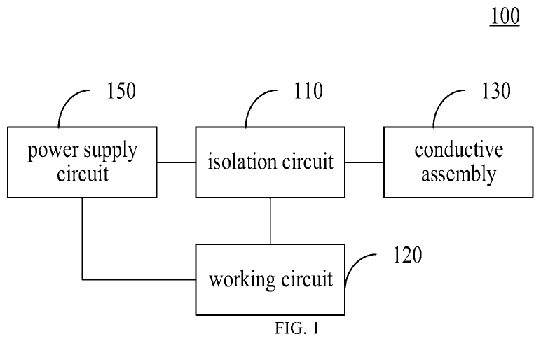

Referring to FIG. 1, the present disclosure provides a beauty device 100. The beauty device 100 includes an isolation circuit 110 and a working circuit 120. A first terminal of the isolation circuit 110 is configured to access a test voltage or output a driving signal. The working circuit 120 is electrically connected to a second terminal of the isolation circuit 110. The working circuit 120 is configured to output the driving signal to the isolation circuit 110.

In the embodiment, the beauty device 100 may further include a conductive assembly 130. The conductive assembly 130 may be a metal electrode. For example, the conductive assembly 130 may include a first electrode plate and a second electrode plate. The first electrode plate and the second electrode plate have opposite polarities. For example, the first electrode plate may be positive and the second electrode plate may be negative; or, the first electrode plate may be negative and the second electrode plate may be positive. The conductive assembly 130 is configured to be in contact with the human skin to provide a micro-current signal, or to access the test voltage. A first terminal of the isolation circuit 110 is electrically connected to the conductive assembly 130. The test voltage may be a high voltage of 1000 V to 1200 V. When testing the beauty device 100, the beauty device 100 is in a power-off state, the isolation circuit 110 is in a disconnected state, and the test voltage may be applied through the conductive assembly 130. Due to the isolation of the isolation circuit 110, the test voltage cannot continue to be transmitted to the working circuit 120 after reaching the isolation circuit 110, such that the high-voltage withstand requirement of the beauty device 100 is met, enhancing the safety of the beauty device 100. When the beauty device 100 works, the isolation circuit 110 is in a conducted state such that the working circuit 120 can output driving signals. The driving signal is transmitted to the conductive assembly 130 through the isolation circuit 110, which ultimately acts on the human skin.

Referring to FIG. 2, in some embodiments, the working circuit 120 may include a control circuit 121 and a driving circuit 122. The control circuit 121 is configured to control the driving circuit 122 to output the driving signal.

When the beauty device 100 works, the control circuit 121 may control the driving circuit 122 to output a driving signal corresponding to a user's setting, and control the corresponding working state (off or on state) of the isolation circuit 110. The control circuit 121 may be a microprocessor, a field programmable gate array (FPGA), or other chips with control function.

In some embodiments, referring to FIG. 2, the driving circuit 122 includes a first switch component Q1, a second switch component Q2, a third switch component Q3, and a fourth switch component Q4. A first terminal of the first switch component Q1, a first terminal of the second switch component Q2, a first terminal of the third switch component Q3, and a first terminal of the fourth switch component Q4 are all electrically connected to the control circuit 121. A second terminal of the first switch component Q1 is configured to access a power supply voltage, a third terminal of the first switch component Q1 is electrically connected to a second terminal of the second switch component Q2, and a third terminal of the second switch component Q2 is grounded. The third terminal of the first switch component Q1 is further electrically connected to the isolation circuit 110. A second terminal of the third switch component Q3 is configured to access the power supply voltage, a third terminal of the third switch component Q3 is electrically connected to a second terminal of the fourth switch component Q4, and a third terminal of the fourth switch component Q4 is grounded. The third terminal of the third switch component Q3 is further electrically connected to the isolation circuit 110. The control circuit 121 is configured to control the first switch component Q1 and the fourth switch component Q4 to be turned on, and to control the third switch component Q3 and the second switch component Q2 to be turned off; or, the control circuit 121 is configured to control the first switch component Q1 and the fourth switch component Q4 to be turned off, and to control the third switch component Q3 and the second switch component Q2 to be turned on.

The control circuit 121 may output a pulse-width modulation (PWM) signal to control the first switch component Q1, the second switch component Q2, the third switch component Q3, and the fourth switch component Q4. For example, when the PWM signal is a high-level signal, the first switch component Q1 and the fourth switch component Q4 are turned on, while the third switch component Q3 and the second switch component Q2 are turned off. In this case, the power supply voltage passes through the first switch component Q1, the first electrode plate, the second electrode plate, and the fourth switch component Q4 to the ground. When the PWM signal is a low-level signal, the first switch component Q1 and the fourth switch component Q4 are turned off, while the third switch component Q3 and the second switch component Q2 are turned on. In this case, the power supply voltage passes through the third switch component Q3, the second electrode plate, the first electrode plate, and the second switch component Q2 to the ground. As such, with the control of the PWM signal, the four switch components are alternately turned on to output a square-wave alternating current signal. The square-wave alternating current signal acts on the human skin after passing through the isolation circuit 110 and the conductive assembly 130.

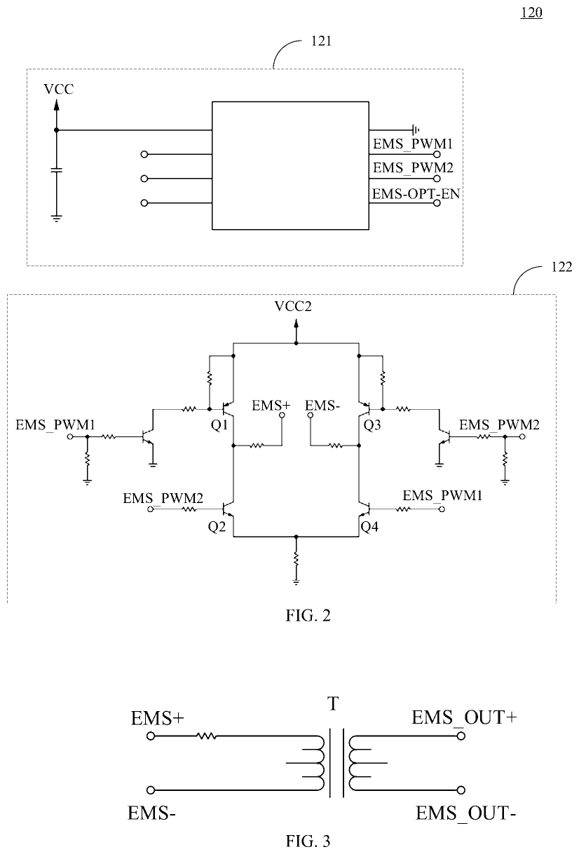

Referring to FIG. 3, in some embodiments, the isolation circuit 110 may include a transformer T. A first terminal of the transformer T is electrically connected to the conductive assembly 130, and a second terminal of the transformer T is electrically connected to the driving circuit 122.

In the embodiments, the transformer T includes a magnetic body and windings on two sides of the magnetic body. During test, the beauty device 100 is in an off state, and the test voltage is applied to the first terminal of the transformer T through the conductive assembly 130. The test voltage is a direct current voltage. Due to the isolation of the transformer T, the test voltage at the first terminal of the transformer T cannot be transmitted to the second terminal, realizing the voltage withstand of the beauty device 100 to the test voltage. In operation, the control circuit 121 controls the driving circuit 122 to output a driving signal. The driving signal is an alternating current signal, and it can be transmitted from the first terminal to the second terminal of the transformer T under the electromagnetic induction of the magnetic body, and then transmitted to the conductive assembly 130. In this case, the working state (off state or on state) of the isolation circuit 110 may not be controlled by the control circuit 121. The transformer T can realize the transmission of the driving signal through electromagnetic induction and provides an electrical isolation for the signal on its ends, enhancing the safety of the beauty device 100. The windings of the transformer T may be wound by means of sandwich winding, making the signal transmission more stable and the waveform more standardized. The magnetic material of the transformer T may be chosen as a silicon steel plate to achieve better low-frequency performance.

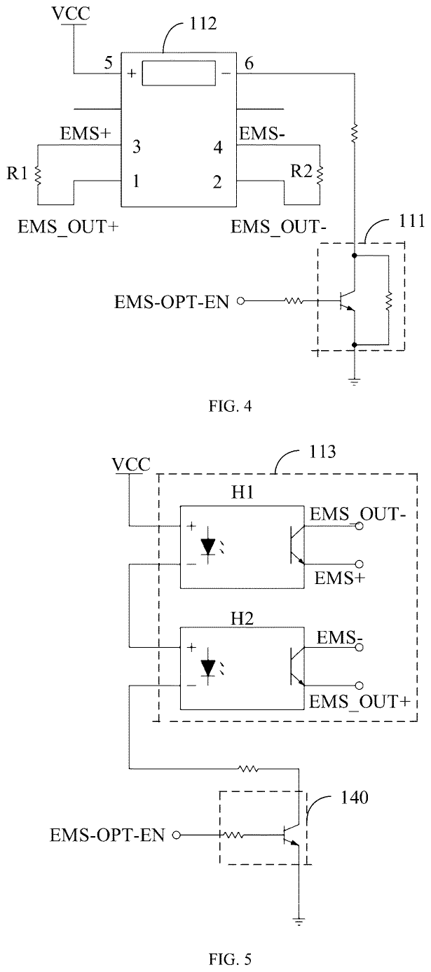

Referring to FIG. 4, in some embodiments, the isolation circuit 110 includes a relay 112. A first terminal of the relay 112 (a pin 1 and a pin 2 of the relay 112 shown in FIG. 4) is electrically connected to the conductive assembly 130, a second terminal of the relay 112 (a pin 3 and a pin 4 of the relay 112 shown in FIG. 4) is electrically connected to the driving circuit 122, and a third terminal of the relay 112 (a pin 5 and a pin 6 of the relay 112 shown in FIG. 4) is electrically connected to the control circuit 121. The control circuit 121 is configured to output a control signal to the third terminal of the relay 112 to control a conduction between the first terminal and the second terminal of the relay 112, to allow the driving circuit 122 to output the driving signal to the first terminal of the relay 112.

In the embodiments, the relay 112 is a normally open relay. The control circuit 121 may output a control signal to control the conduction between the first terminal and the second terminal of the relay 112, to allow the driving signal output by the driving circuit 122 to be transmitted to the conductive assembly 130 through the relay 112. During test, the beauty device 100 is in an off state, no control signal is applied to the third terminal of the relay 112, and the first terminal and the second terminal of the relay 112 remain in a disconnected state. There is no transmission path for the test voltage after it being transmitted through the conductive assembly 130 to the first terminal of the relay 112, thus the test voltage cannot be transmitted to the second terminal of the relay 112, realizing the voltage withstand to the test voltage. During operation, the control circuit 121 may output the control signal according to a user's setting, to control the conduction between the first terminal and the second terminal of the relay 112, realizing a normal transmission of the driving signal. The relay 112 may be a relay selected based on actual requirements of voltage withstand performance, ensuring the voltage withstand requirement of the relay 112.

In some embodiments, the beauty device 100 further includes a first resistor R1 and a second resistor R2. The first terminal of the relay 112 includes a first pin (namely the pin 1 shown in FIG. 4) and a second pin (namely the pin 2 shown in FIG. 4). The second terminal of the relay 112 includes a third pin (namely the pin 3 shown in FIG. 4) and a fourth pin (namely the pin 4 shown in FIG. 4). Two ends of the first resistor R1 are electrically connected to the first pin and the third pin respectively, and two ends of the second resistor R2 are electrically connected to the second pin and the fourth pin respectively. The control circuit 121 is configured to output the control signal to the third terminal of the relay 112 to control a conduction between the first pin and the third pin and a conduction between the second pin and the fourth pin, and allow the driving circuit 122 to output the driving signal to the third pin and the fourth pin.

In the embodiments, the third pin and the fourth pin of the relay 112 are respectively configured to receive an EMS+ signal and an EMS− signal output by the driving circuit 122. When the beauty device 100 works, the control circuit 121 outputs the control signal to control a conduction between the first pin and the third pin of the relay 112, and a conduction between the second pin and the fourth pin of the relay 112. The EMS+ signal is transmitted to the first pin through the third pin, and reaches the human skin through the first electrode plate. The EMS− signal is transmitted to the second pin through the fourth pin, and reaches the human skin through the second electrode plate.

During test, the beauty device 100 is in an off state, and no control signal is applied to the third terminal of the relay 112. The first pin and the third pin of the relay 112 remain disconnected, the second pin and the fourth pin also remain disconnected. After the test voltage is transmitted to the first pin and the second pin of the relay 112 through the conductive assembly 130, the transmission path is disconnected, and the test voltage cannot be transmitted to the second terminal of the relay 112, realizing the withstand of the beauty device to the test voltage.

In some embodiments, as shown in FIG. 4, the isolation circuit 110 further includes a first switch circuit 111. The third terminal of the relay 112 includes a fifth pin (namely the pin 5 shown in FIG. 4) and a sixth pin (namely the pin 6 shown in FIG. 4). The fifth pin is configured to access a power supply voltage, and the sixth pin is electrically connected to a first terminal of the first switch circuit 111. A second terminal of the first switch circuit 111 is electrically connected to the control circuit 121, and a third terminal of the first switch circuit 111 is grounded. The control circuit 121 is configured to control a conduction between the first terminal and the third terminal of the first switch circuit 111, to control the conduction between the first terminal and the second terminal of the relay 112.

In the embodiments, the first switch circuit 111 may be a triode. The relay 112 may further include a magnetic core, two ends of which are the fifth pin and the sixth pin respectively. When the beauty device 100 works, the control circuit 121 may output a high/low level to control the conduction between the first terminal and the third terminal of the first switch circuit 111, to allow the power supply voltage to be transmitted from the magnetic core to the ground through the first switch circuit 111. A current flows through the magnetic core to generate a magnetic field which attracts the contact points of the first pin and the third pin to close and the contact points of the second pin and the fourth pin to close, thereby conducting the driving circuit 122 with the conductive assembly 130.

During test, the beauty device 100 is in an off state, no power supply voltage is applied to the fifth pin of the relay 11. The first pin and the third pin of the relay 112 are disconnected, and the second pin and the fourth pin are also disconnected, achieving the voltage withstand to the test voltage.

In some embodiments, the isolation circuit 110 may further include a third optocoupler, a fourth optocoupler, and a third switch circuit. The third optocoupler and the fourth optocoupler are arranged between the conductive assembly 130 and the relay 112, and configured to realize a connection and control between the relay 112 and the conductive assembly 130. The third switch circuit may be a triode. A first terminal of the third optocoupler is configured to access a power supply voltage, a second terminal of the third optocoupler is electrically connected to a first terminal of the fourth optocoupler, a second terminal of the fourth optocoupler is electrically connected to a first terminal of the third switch circuit, a second terminal of the third switch circuit is a controlled terminal, and a third terminal of the third switch circuit is grounded. In this way, by controlling the third switch circuit on or off, the third optocoupler and the fourth optocoupler can be controlled to be conducted or disconnected to realize the transmission of the driving signal.

In some embodiments, referring to FIG. 5, the isolation circuit 110 includes an isolation optocoupler 113. A first terminal of the isolation optocoupler 113 is electrically connected to the conductive assembly 130, and a second terminal of the isolation optocoupler 113 is electrically connected to the driving circuit 122. The driving circuit 122 is configured to output the driving signal to the first terminal of the isolation optocoupler 113. A third terminal of the isolation optocoupler 113 is electrically connected to the control circuit 121.

In the embodiments, when the beauty device 100 works, the control circuit 121 may control a conduction of the isolation optocoupler 113 to transmit the driving signal output by the driving circuit 122 to the conductive assembly 130. During test, the beauty device 100 is in an off state, and the isolating optocoupler 113 is disconnected. The test voltage is applied through the conductive assembly 130 and cannot continue to be transmitted to the second terminal of the isolation optocoupler 113 after reaching the first terminal of the isolation optocoupler 113, achieving the voltage withstand to the test voltage. The isolation optocoupler 113 may be an optocoupler with a high-voltage withstand performance.

In some embodiments, the beauty device 100 further includes a second switch circuit 140. The third terminal of the isolation optocoupler 113 is electrically connected to the control circuit 121 by the second switch circuit 140. A first terminal of the second switch circuit 140 is electrically connected to the third terminal of the isolation optocoupler 113, a second terminal of the second switch circuit 140 is grounded, and a third terminal of the second switch circuit 140 is electrically connected to the control circuit 121. The control circuit 121 is configured to control a conduction between the first terminal and the second terminal of the second switch circuit 140, to control the isolation optocoupler 113 to transmit the driving signal.

In the embodiments, the second switch circuit 140 may be a triode. When the beauty device 100 works, the control circuit 121 may control the conduction between the first terminal and the second terminal of the second switch circuit 140 to conduct the isolation optocoupler 113, realizing the transmission of the driving signal.

In some embodiments, the isolation optocoupler 113 includes a first optocoupler H1 and a second optocoupler H2. A first terminal of the first optocoupler H1 is configured to access a power supply voltage, a second terminal of the first optocoupler H1 is electrically connected to a first terminal of the second optocoupler H2, a third terminal of the first optocoupler H1 is electrically connected to the driving circuit 122, and a fourth terminal of the first optocoupler H1 is electrically connected to the conductive assembly 130. A second terminal of the second optocoupler H2 is electrically connected to the first terminal of the second switch circuit 140, a third terminal of the second optocoupler H2 is electrically connected to the driving circuit 122, and a fourth terminal of the second optocoupler H2 is electrically connected to the conductive assembly 130. The second terminal of the isolation circuit 110 includes the third terminal of the first optocoupler H1 and the third terminal of the second optocoupler H2.

In the embodiments, the first optocoupler H1 may be a metal-oxide-semiconductor (MOS) field-effect transistor with a high-voltage withstand performance. For example, the first optocoupler H1 may include two MOS transistors with a withstand voltage of 1500 V. The two MOS transistors are connected in series, and electrically connected to the conductive assembly 130 and the driving circuit 122. In this way, under a voltage stress of 0.8, the two MOS transistors with the withstand voltage of 1500 V can reach a withstand voltage of 1200 V, realizing the voltage withstand of the beauty device 100 to the test voltage. The second optocoupler H2 has the same structure as the first optocoupler H1.

When the beauty device 100 works, the control circuit 121 controls the second switch circuit 140 to be turned on, and the power supply voltage passes through the first terminal of the first optocoupler H1, the second terminal of the first optocoupler H1, the first terminal of the second optocoupler H2, and the second terminal of the second optocoupler H2 to the ground, to conduct the third terminal and the fourth terminal of the first optocoupler H1, and conduct the third terminal and the fourth terminal of the second optocoupler H2, realizing the transmission of the driving signal. During test, the beauty device 100 is in an off state, the first optocoupler H1 and the second optocoupler H2 are both disconnected. The test voltage reaches the fourth terminal of the first optocoupler H1 and the fourth terminal of the second optocoupler H2 through the conductive assembly 130. Due to the voltage withstand performance of the first optocoupler H1 and the second optocoupler H2, the test voltage will not break the first optocoupler H1 and the second optocoupler H2, realizing the voltage withstand to the test voltage.

In some embodiments, referring to FIG. 1 again, the beauty device 100 may further include a power supply circuit 150. The power supply circuit 150 is configured to output a power supply voltage to the isolation circuit 110 and the working circuit 120. The control circuit 121 is further configured to control the driving circuit 122 to convert the power supply voltage to the driving voltage for output.

In the embodiments, the power supply circuit 150 may be a boost/buck converter chip. An input terminal of the power supply circuit 150 may be connected to a battery or a mains electricity supply. The power supply circuit 150 may provide the power supply voltage to the driving circuit 122 and the control circuit 121, to allow the control circuit 121 to control the driving circuit 122 to output the driving signal. The power supply circuit 150 may also provide the power supply voltage to the isolation circuit 110, to allow the control circuit 121 to control the isolation circuit 110 to operate.

In the present disclosure, the voltage withstand to the test voltage is realized by the isolation of the isolation circuit 110, so as to meet the voltage withstand requirement of the beauty device 100 and improve the safety of the beauty device 100. The isolation circuit 110 can also realize the transmission of the driving signal, ensuring the normal operation of the beauty device 100.

The working principle of the beauty device is to use technologies such as micro-frequency current, radio frequency, ultrasound, and intense light to stimulate and act on the skin, to improve skin metabolism and contract muscles, thus achieving cosmetic effects. When using radio frequency for skin care, electrodes may be provided on the beauty device. During the operation of the beauty device, the electrodes are in contact with the skin and output a radio frequency current to generate a thermal effect, thereby achieving the cosmetic effects.

In related art, the beauty device cannot generate a uniform heat distribution on the skin due to the shape and layout of the electrodes, which leads to a non-uniform effect of the beauty device, causing regional differences on skin beautification.

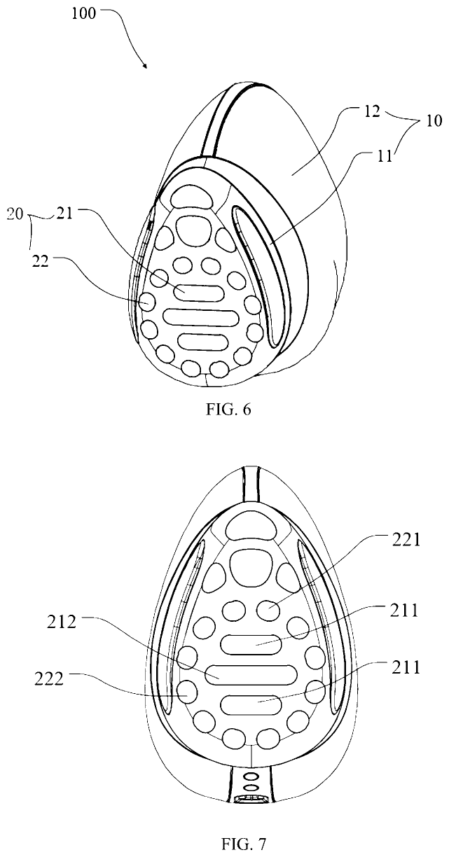

In some embodiments, referring to FIG. 6 to FIG. 10, the beauty device 100 further includes a housing 10 and an electrode assembly 20 arranged in the housing 10. The electrode assembly 20 is at least partially exposed outside the housing 10 for contacting a user's skin to output energy such as radio frequency energy to the contacted skin area, to achieve cosmetic effects such as wrinkle reduction and skin tightening.

In the embodiments, the housing 10 includes an end portion 11 for contacting a to-be-treated skin and a side portion 12 for hand-holding. The electrode assembly 20 is arranged at the end portion 11 of the housing 10. When using the beauty device 100, a user may hold the side portion 12 and place the electrode assembly 20 against the skin. Additionally, while the electrode assembly 20 is in contact with the skin, the user may move the beauty device 100 across the skin surface to provide skincare.

A radio frequency generator (not shown) is arranged in the housing 10. A radio frequency current is generated by the radio frequency generator and transmitted to the electrode assembly 20. By way of the electrode assembly 20, the generated radio frequency current acts on subcutaneous tissues such as elastic fibers and collagen in the skin. These subcutaneous tissues contract after being heated, thus achieving skin tightening. Additionally, the thermal stimulation can promote the regeneration and remodeling of new collagen fibers and collagen.

The electrode assembly 20 is at least partially exposed outside the housing 10, for example, the electrode assembly 20 is protruded outside the housing 10, the electrode assembly 20 is flush with the surface of the housing 10, or the electrode assembly 20 is recessed a certain distance from the surface of the housing 10. The present disclosure has no limitation thereto, as long as the electrode assembly 20 can output the radio frequency current to the skin during the process of the housing 10 contacting the skin.

It should be understood that the technical solutions of some embodiments of the present disclosure are improvements made to the arrangement of the electrode assembly 20. The other parts and related functions of the beauty device 100 of the present disclosure are all known in related art, which is not detailed herein. Those skilled in the art should be aware of and proficient in these aspects.

The electrode assembly 20 includes a first electrode group 21 and a second electrode group 22 arranged around the first electrode group 21. The second electrode group 22 is disposed around the first electrode group 21 to form a circular ring, an elliptical ring, or other shapes, so as to facilitate the formation of a large-area of treatment between the second electrode group 22 and the first electrode group 21.

The first electrode group 21 includes a first electrode 211 and a second electrode 212 that are spaced apart from each other. The first electrode 211 and the second electrode 212 have opposite polarities and form an electrode pair. When the first electrode 211 and the second electrode 212 are in contact with the skin at the same time, they may form a circuit to allow the radio frequency current to circulate to act on the user's skin and provide an internal heating. This is beneficial for lifting and tightening the skin, enhancing skin elasticity, thus being conducive to achieving the cosmetic effects of reducing wrinkles.

A part of electrodes in the second electrode group 22 has a polarity opposite to that of the first electrode 211 and form an electrode pair with the first electrode 211. When the two are in contact with the skin at the same time, they may form a circuit to allow the the radio frequency current to circulate to act on the user's skin. The other part of the electrodes in the second electrode group 22 has a polarity opposite to that of the second electrode 212 and form an electrode pair with the second electrode 212. When the two are in contact with the skin at the same time, they may form a circuit to allow the radio frequency current to circulate to act on the user's skin. This stimulates the skin to achieve the effects of lifting and tightening the skin, as well as effective anti-aging.

In this way, a heating area may be formed between the first electrode 211 and the second electrode 212, and there is also a heating area formed between the second electrode group 22 and the first electrode 211, and between the second electrode group 22 and the second electrode 212. Compared to the beauty device in related art including two concentric ring-shaped electrodes and only forming a ring-shaped heat distribution between the two ring-shaped electrodes, the heating area of the entire electrode assembly 20 of the beauty device 100 is increased. Moreover, the larger area inside the inner ring electrode of the beauty device in related art does not produce a discharge heat effect, which results in an inability to form a uniform heat distribution on the skin. However, the beauty device 100 of the present embodiment has a more uniform heat distribution throughout the electrode assembly 20, thereby forming a uniform heat distribution on the skin, ensuring a uniform effect of the beauty device 100, and preventing a regional difference on skin beautification.

It can be understood that the first electrode 211 may be positive, and correspondingly, the second electrode 212 may be negative. That is, the first electrode 211 and the second electrode 212 have opposite polarities. When the two are in contact with the skin at the same time, a circuit may be formed. A radio frequency current flows from the first electrode 211 toward the second electrode 212 and acts on the contacted skin area to provide skincare. In some other embodiments, the first electrode 211 may be negative, and the second electrode 212 may be positive, such that the two have opposite polarities to form a circuit, facilitating the circulation of the radio frequency current.

The first electrode 211 and the second electrode 212 are both in the shape of a strip. The first electrode 211 and the second electrode 212 are parallel to each other, that is, the first electrode 211 and the second electrode 212 extend in the same direction. When the beauty device 100 moves along the skin surface, the first electrode 211 and the second electrode 212 are in contact with the skin at the same time to form a circuit, and a radio frequency current is generated at the contacted skin area to provide skincare.

In some embodiments, the first electrode 211 and the second electrode 212 extend along the length or width direction of the end portion 11 of the housing 10. In the embodiments, the first electrode 211 and the second electrode 212 extend along the width direction of the end portion 11 of the housing 10. When the beauty device 100 is pushed on the skin surface, the length direction of the first electrode 211 and the length direction of the second electrode 212 are consistent with the moving direction of the beauty device 100, which reduces the sensation of foreign matter on the user's skin during the beauty device 100 is in operation, achieving a more comfortable care experience and a higher user experience.

In some other embodiments, the first electrode 211 and the second electrode 212 may be provided with other shapes, such as a rectangular shape, a fan shape, or a bow-like shape, which is not limited herein. In addition, the first electrode 211 or the second electrode 212 may refer to a continuous electrode, which is set in the shape of a bar, a rectangle, a fan, or a bow. Or, the first electrode 211 or the second electrode 212 may refer to at least two sub-electrodes consisting the first electrode 211 or the second electrode 212, and the whole of the at least two sub-electrodes is set in the shape of a bar, a rectangle, a fan, or a bow. The shape of each sub-electrode is not limited herein, for example, the sub-electrode may be a circle or an arc.

In some embodiments, referring to FIG. 10, one first electrode 211 and one second electrode 212 are provided. The first electrode 211 and the second electrode 212 have opposite polarities and form an electrode pair. When both the first electrode 211 and the second electrode 212 are in contact with the skin, the two electrodes may form a circuit to allow the radio frequency current to circulate to act on the user's skin. This stimulates the skin, achieving the effects of lifting and tightening the skin, as well as effective anti-aging.

As described above, the present disclosure does not limit the shapes of the first electrode 211 and the second electrode 212. It can be understood that, in a configuration that both the first electrode 211 and the second electrode 212 have a bow-like shape, the first electrode 211 and the second electrode 212 are arranged in mirror symmetry.

In some other embodiments, referring to FIG. 7, two first electrodes 211 are provided, and the second electrode 212 is arranged between the two first electrodes 211. The second electrode 212 and each adjacent first electrode 211 form an electrode pair. In a configuration that the second electrode group 22 is arranged around the first electrode group 21 to form a circle, the multiple first electrodes 211 are spaced apart from the second electrode 212, which makes more full and rational use of the space within the circle, facilitating a more uniform distribution of heat.

In some embodiments, the second electrode group 22 includes a third electrode 221 provided corresponding to the first electrode 211. The third electrode 221 is arranged adjacent to the first electrode 211. The third electrode 221 and the first electrode 211 have opposite polarities and form an electrode pair. When the two are in contact with the skin at the same time, they may form a circuit to allow the radio frequency current to circulate to act on the user's skin. This stimulates the skin, achieving the effects of lifting and tightening the skin, as well as effective anti-aging.

It can be understood that, in a configuration that one first electrode is provided (e.g., as shown in FIG. 10), then one third electrode 221 is provided; and in a configuration that two first electrodes are provided (e.g., as shown in FIG. 6), then two third electrodes 221 are provided, and each third electrode 221 is arranged adjacent to one first electrode 211. It should be noted that the third electrode 221 may refer to a continuous electrode with an arc shape; or, the third electrode 221 may refer to at least two sub-electrodes consisting the third electrode 221, and the whole third electrode 221 formed by the at least two sub-electrodes is in an arc shape. The shape of the sub-electrode is not limited, for example, the sub-electrode may also be dot-shaped or arc-shaped.

In some embodiments, referring to FIG. 7 again, there are a plurality of sub-electrodes consisting the third electrode 221, and each sub-electrode is in a dot shape, whose cross section may be circular or elliptical. In the embodiments, the third electrode 221 includes multiple sub-electrodes, and the sub-electrodes are arranged at intervals in the vicinity of the first electrode 211. The third electrode 221 and the first electrode 211 have opposite polarities and form an electrode pair, thereby forming a circuit to act on the skin.

Compared to using an electrode with a large-area for discharge, each the dot-shaped third electrode 221 has a smaller individual area, but the whole effective contact area of the multiple third electrodes 221 with the skin are larger. For example, in curved areas such as the lower jaw, the third electrodes 221, which are set in a dotted pattern, facilitates contact with the skin compared to the electrode with a large-area. In addition, the dotted third electrodes 221 may be arranged at intervals to further enlarge the contacted region of the skin and improve the uniform of the heat distribution.

In some other embodiments, referring to FIG. 9, two third electrodes 221 are provided, each of the third electrode 221 is provided with a shape of arc. In the embodiments, each third electrode 221 is arranged adjacent to one first electrode 211. Each third electrode 221 and its adjacent first electrode 211 have opposite polarities and form an electrode pair, thereby forming a circuit to act on the skin.

Compared to the dot-shaped third electrodes 221, the arc-shaped third electrodes 221 in the embodiments help to reduce the number of electrodes and make the assembly of the third electrodes more convenient and faster.

In some other embodiments, the third electrode 221 may include a combination of dot-shaped sub-electrodes and arc-shaped sub-electrodes, which may be specifically set according to actual needs to achieve the optimal skin treatment effect.

In some embodiments, the second electrode group 22 further includes a fourth electrode 222 provided corresponding to the second electrode 212. The fourth electrode 222 is arranged adjacent to the second electrode 212. The fourth electrode 222 and the second electrode 212 have opposite polarities and form an electrode pair. When both the fourth electrode and the second electrode are in contact with the skin, they may form a circuit to allow the radio frequency current to circulate to act on the user's skin. This stimulates the skin, achieving the effects of lifting and tightening the skin, as well as effective anti-aging. For the definition of “one fourth electrode 222”, please refer to the aforementioned definition of “one third electrode 221” and do not repeat it herein.

In the embodiments as shown in FIG. 7, there are two third electrodes 221, and each third electrode 221 includes four sub-electrodes and is arranged close to one first electrode 211; there are two fourth electrodes 222, and each fourth electrode 222 includes two sub-electrodes and is arranged close to the second electrode 212. Each fourth electrode 222 is arranged adjacent to a corresponding third electrode 221 to form an electrode pair. In the embodiments, the electrodes are evenly distributed, realizing that the output energy is uniformly distributed, prompting the energy acting on the skin to be uniformly distributed, and making the deep heating effect more obvious.

In some other embodiments, the number of the first electrodes 211, the second electrodes 212, the third electrodes 221, and the fourth electrodes 222 may be set according to actual requirements of users, which is not limited herein. Moreover, a user may adjust the mode of the beauty device 100 to control the number of discharging electrode pairs, to control the intensity of the beauty device 100, thereby preventing skin burns.

In some embodiments, the third electrode 221 and the fourth electrode 222 have opposite polarities. The third electrode 221 and its adjacent fourth electrode 222 form an electrode pair. When both the two electrodes are in contact with the skin, a circuit may be formed to allow the radio frequency current to circulate to act on the user's skin. This stimulates the skin, achieving the effects of lifting and tightening the skin, as well as effective anti-aging.

In the embodiments, the fourth electrode 222 is arranged between the two third electrodes 221. The third electrode 221 and its adjacent fourth electrode 222 have opposite polarities and form an electrode pair. This maximizes the utilization of the area of the end portion 11 of the housing 10, thereby increasing the heating area.

Specifically, the fourth electrode 222 includes a plurality of sub-electrodes, each sub-electrode is provided with a dot shape. Two fourth electrodes 222 are arranged on two sides of the second electrode 212 to form electrode pairs with the second electrode 212. In some other embodiments, the fourth electrode 222 may be in other shapes, such as an arc shape, which is not limited herein.

In some embodiments, a length of the second electrode 212 is greater than a length of the first electrode 211 located on either side of the second electrode 212. In a configuration that the second electrode group 22 is arranged around the first electrode group 21 to enclose a circle, the first electrode group 21 is arranged at the center of the circle, and the length of the second electrode 212 is greater than the length of the first electrode 211 located on either side of the second electrode 212. That is, the two ends of the second electrode 212 protrude beyond the two ends of the first electrode 211 located on either side of the second electrode 212. This is beneficial for the fourth electrode 222 to be closer to the second electrode 212 and form an electrode pair with the second electrode 212, forming a circuit to allow the radio frequency current to circulate to act on the user's skin to achieve the effects of lifting and tightening the skin, as well as effective anti-aging.

In some embodiments, the third electrode 221 and the fourth electrode 222 are arranged spaced apart and around two first electrodes 211 and the second electrode 212, the second electrode 212 is arranged at the center of the circle enclosed by the third electrode 221 and the fourth electrode 222, and the two first electrodes 211 are symmetrically arranged on the two sides of the second electrode 212. This arrangement can fully and reasonably utilize the space of the end portion 11 of the housing 10, resulting in a large heating area with a uniform heat distribution.

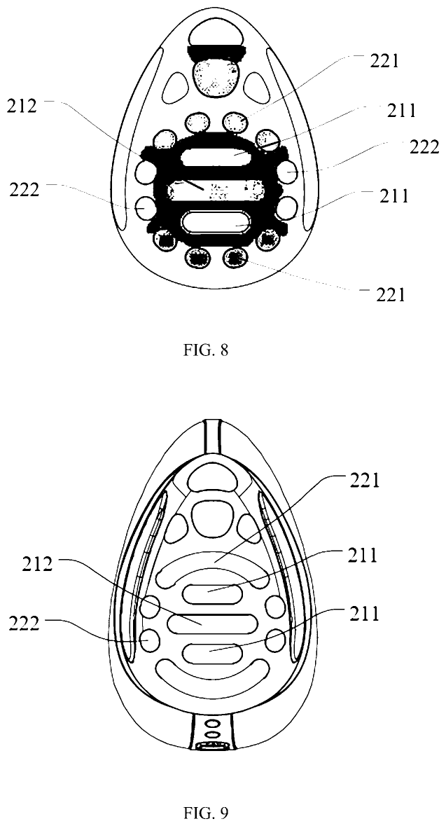

Referring to FIG. 8, which is a heat distribution diagram of the electrode assembly 20 in FIG. 6. The gray part may indicate the positive electrodes (e.g., the second electrode 212 and the third electrode 221), and the light gray part may indicate the negative electrodes (e.g., the first electrode 211 and the fourth electrode 222). A heating area (the dark gray part) is formed between the positive electrode and the negative electrode. The heating area is relatively large and can provide a uniform heat distribution on the skin, which ensures a uniform effect of the beauty device 100 to the skin, preventing regional differences on skin beautification.

In some other embodiments, the gray part may indicate the negative electrodes, and the light gray part may indicate the positive electrodes.

In the embodiments as shown in FIG. 10, there are one third electrode 221 arranged adjacent to the first electrode 211 and one fourth electrode 222 arranged adjacent to the second electrode 212. The number of sub-electrodes of the third electrode 221 may be equal to the number of sub-electrodes of the fourth electrode 222, for example, the number is 6. In the embodiments, the electrodes are evenly distributed, realizing that the output energy is uniformly distributed, prompting the energy acting on the skin to be uniformly distributed, and making the deep heating effect more obvious.

In some other embodiments, the numbers of the first electrodes 211, the second electrodes 212, the third electrodes 221, and the fourth electrodes 222 may be set according to actual requirements of users, which is not limited herein. Moreover, a user may adjust the mode of the beauty device 100 to control the number of discharging electrode pairs, to control the intensity of the beauty device 100, thereby preventing skin burns.

The second electrode group 22 includes one third electrode 221. the third electrode 221 includes a plurality of sub-electrodes arranged at intervals and adjacent to the first electrode 211. The third electrode 221 and the first electrode 211 have opposite polarities and form an electrode pair. When the two electrodes are in contact with the skin at the same time, a circuit may be formed to allow the radio frequency current to circulate to act on the user's skin.

The second electrode group 22 further includes one fourth electrode 222. The fourth electrode 222 includes a plurality of sub-electrodes arranged at intervals and adjacent to the second electrode 212. The fourth electrode 222 and the second electrode 212 have opposite polarities and form an electrode pair. When the two electrodes are in contact with the skin at the same time, a circuit may be formed to allow the radio frequency current to circulate to act on the user's skin.

In the embodiments, the third electrode 221 and the fourth electrode 222 may be in a dot shape or an arc shape, and are arranged along the periphery of the bowed surfaces of the first electrode 211 and the second electrode 212 respectively, so as to form an electrode pair with the first electrode 211 and the second electrode 212, respectively.

According to the beauty device 100 provided in the present disclosure, the first electrode group 21 includes the first electrode 211 and the second electrode 212 spaced apart from the first electrode 211, the first electrode 211 and the second electrode 212 have opposite polarities to form an electrode pair. A part of electrodes in the second electrode group 22 has a polarity opposite to that of the first electrode 211 and form an electrode pair with the first electrode 211, and the other part of the electrodes in the second electrode group 22 has a polarity opposite to that of the second electrode 212 and form an electrode pair with the second electrode 212. In this way, a heating area can be formed between the first electrode 211 and the second electrode 212, between the second electrode group 22 and the first electrode 211, and between the second electrode group 22 and the second electrode 212, which increases the heating area of the entire electrode assembly 20, to form a uniform heat distribution on the skin. This ensures a uniform treatment effect of the beauty device 100, preventing causing regional differences on skin beautification.

The working principle of the beauty device is to use technologies such as micro-frequency current, radio frequency, ultrasound, and intense light to stimulate and act on the skin, to improve skin metabolism and contract muscles, achieving cosmetic effects. For example, a radio frequency beauty device uses a radio frequency energy for skin treatment. For another example, a microcurrent beauty device uses a microcurrent for skin treatment. This type of radio frequency or microcurrent beauty device mainly includes an electrode head that comes into contact with the skin and a control apparatus. The electrode head is controlled to generate a radio frequency signal or a microcurrent to stimulate the user's skin, achieving cosmetic effects. The radio frequency or microcurrent beauty device is generally provided with an indicator lamp to indicate the working state of the beauty device.

The beauty device in the related art generally uses a light guide component to cover the outside of the indicator lamp, and the light guide component in related art is a transparent component, which can guide all the light emitted by the indicator lamp. Since the light guide component causes light loss during the transmission process, the intensity of the light transmitted through the light guide component is insufficient, which affects the user's observation of the state of the indicator lamp. Therefore, in order to solve the above technical problem, the present disclosure proposes at least the following embodiments.

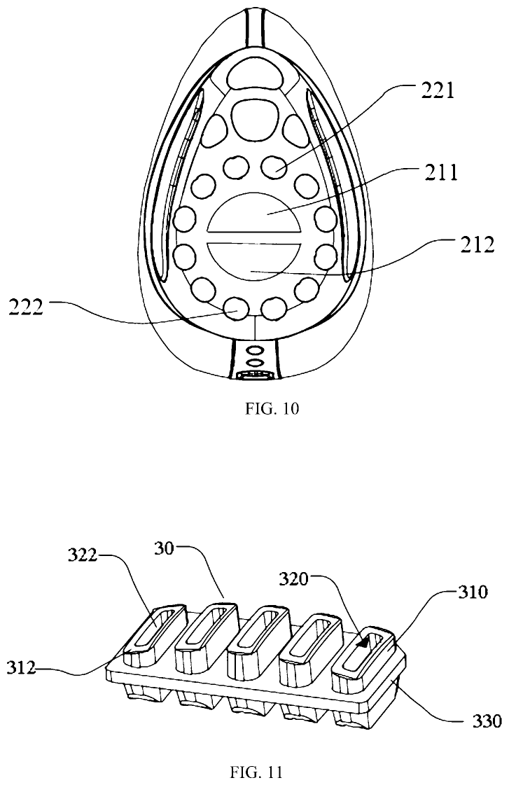

In some embodiments, the beauty device 100 further includes a light guide component 30. Referring to FIG. 11 and FIG. 13, in some embodiments, the light guide component 30 includes a side wall 310. An end face 311 of the side wall 310 may be configured to allow light of the indicator lamp of the beauty device to enter, and the other end face 312 of the side wall 310 may be configured to allow the light of the indicator lamp of the beauty device to exit. The light of the indicator lamp of the beauty device enters from the end face 311 of the side wall 310, passes through the side wall 310, and exits from the other end face 312 of the side wall 310.

In some embodiments, the side wall 310 is made of a light guide material, including but not limited to polymer materials (such as polystyrene), a glass light guide plate, and other light guide materials. In some other embodiments, the side wall 310 may be made of any material that emits light when illuminated.

In the embodiments, the end face 311 and the other end face 312 of the side wall 310 face each other to facilitate the entrance and exit of light.

When the light of the beauty device 100 enters the side wall 310 from the end face 311 of the side wall 310, the side wall 310 scatters the light emitted by the indicator lamp of the beauty device to form a uniform light, and allows the uniform light to exit from the other end face 312 of the side wall 310. Thus, the light exiting from the side wall 310 is more evenly distributed.

The light guide component 30 further includes a channel 320 defined by the side wall 310. One end of the channel 320 is opened to define a light inlet 321 for the light of the indicator lamp of the beauty device to enter, and the other end of the channel 320 is opened to define a light outlet 322 for the light of the indicator lamp of the beauty device to exit. The light of the indicator lamp of the beauty device enters from the light inlet 321, passes through the channel 320, and exits from the light outlet 322.

In the embodiments, the light inlet 321 faces the light outlet 322, allowing the light of the indicator lamp of the beauty device to enter and exit, respectively. Since the light emitted by the indicator lamp of the beauty device is transmitted without obstruction in the channel 320, the loss of light transmission can be greatly reduced, so that the intensity of the light emitted through the channel 320 can be strengthened.

In the embodiments, by arranging the side wall 310 and the channel 320 defined by the side wall 310, the light emitted by the indicator lamp of the beauty device is divided into two parts to enter the light guide component 30: a part of the light enters from the end face 311 of the side wall 310 and exits from the other end face 312 of the side wall 310; the other part of the light enters from the light inlet 321 of the channel 320 and exits from the light outlet 322, such that a part of the light emitted by the indicator lamp of the beauty device is guided out through the side wall 310, and the other part of the light directly exits through the channel 320. This ensures the intensity of the light while guiding the light, enhancing the indication effect of the indicator lamp of the beauty device and making it easier for users to observe the working state of the beauty device.

The light guide component 30 according to the embodiments is small in size and can be mounted on the side surface of the beauty device 100. By way of the light-guiding and light-transmitting functions, the light guide component 30 help to enhance the uniformity and brightness of the light emitted, making it easier for users to observe the working state of the beauty device. This solves the problem in the related art that the indicator lamp, when mounted on the side surface of the beauty device, does not provide sufficient light guidance intensity.

In some embodiments, the light guide component 30 is a cylindrical structure, and the channel 320 is defined in the cylindrical structure. That is, the light guide component 30 is a hollow structure, and the inner wall of the channel 320 allows the light to enter. In this way, a part of the light of the indicator lamp of the beauty device exits through the side wall 310; the other part of the light enters the channel 320, and the light emitting through the channel 320 partially enters the side wall 310 of the light guide component 30 from the inner wall of the channel 320 and exits through the side wall 310. This increases the light exported from the side wall 310, allowing the light guide component 30 to balance the intensity and uniformity of the emitted light.

In some other embodiments, the light guide component 30 may be other structures, such as a flared structure, which is not limited herein. In a configuration that the light guide component 30 is a flared structure, a part of the light emitted by the indicator lamp of the beauty device is guided out through the side wall 310, and the other part of the light exits through the channel 320. Moreover, part of the light enters the side wall 310 while the light passes through the channel 320. The incident area of the light from the channel 320 into the light guide component 30 increases compared with that of the channel of the cylindrical light guide component 30, which further enhances the intensity of the light emitted from the side wall 310, and improves the overall light-emitting effect of the light guide component 30.

In some embodiments, at least an inner end of the side wall 310 close to where the light exits is provided with a reflective layer. In other words, the reflective layer is arranged at the end of the side wall 310 away from the indicator lamp of the beauty device.

In the embodiments, the reflective layer may be made of a retro-reflective material that has already been processed into a thin film and is ready for use, and is configured to reflect the light emitted by the indicator lamp of the beauty device. In this way, a part of the light emitted by the indicator lamp of the beauty device may be guided out at the end of the side wall 310 close to the indicator lamp; the part of the light exits through the channel 320 and is reflected by the reflective layer and then exits. This reduces light loss, further enhancing the brightness of the emitted light.

In some embodiments, the outer surface of the reflective layer is a curved surface or an uneven surface. Any chemical or physical process can be utilized to create a reflective surface on the curved or uneven surface, including methods such as metal coating or the application of polymer coating on the curved surface or the uneven surface.

According to the embodiments, by providing the reflective layer with a curved surface or an uneven surface, the light can be reflected multiple times, thereby enhancing the reflective effect. This allows for a more uniform brightness of the emitted light and an improved light diffusion effect.

In some embodiments, the uneven surface is a wave-shaped surface. By replacing the conventional flat surface with the uneven surface, the light emitted by the indicator lamp of the beauty device can be reflected multiple times at different angles, such that the light will be mixed more uniformly after it is emitted, improving the effect of the light.

In some other embodiments, the uneven surface may be in other shapes, which is not limited herein.

In some embodiments, the end face 311 of the side wall 310 for the light of the indicator lamp of the beauty device to enter is inclined.

In the embodiments, the end face 311 of the side wall 310 for the light of the indicator lamp of the beauty device to enter is inclined downward from the inside to the outside. Compared to a parallel arrangement, the inclined end face 311 can increase the area of light introduction, thereby improving the light guiding efficiency and enhancing the brightness of the emitted light. Moreover, the end face 311 that is inclined downward from the inside to the outside can gather the light and prevent it from diverging.

In some other embodiments, the end face 311 of the side wall 310 for the light of the indicator lamp of the beauty device to enter may be inclined upward from the inside to the outside, which is not limited herein.

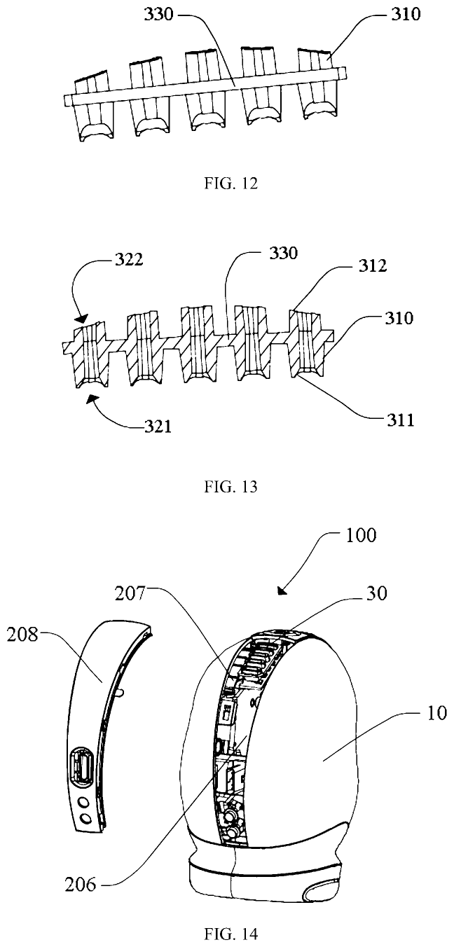

Referring to FIG. 12, in some embodiments, there are a plurality of light guide components 30 arranged at intervals and corresponding to a plurality of indicator lamps of the beauty device. The plurality of indicator lamps of the beauty device may correspond to different working states of the beauty device 100, such as a remaining power, a working mode, or a working duration of the beauty device 100. The plurality of light guide components 30 are arranged at intervals and corresponding to the plurality of indicator lamps of the beauty device, so as to realize respective independent light-guiding effects without affecting each other. This facilitates the user to identify and promptly understand the different working states of the beauty device 100.

In some embodiments, referring to FIG. 12 and FIG. 13, adjacent light guide components 30 are connected by a connecting component 330. In the embodiments, the plurality of light guide components 30 are connected by the connecting component 330, such that the plurality of light guide components 30 can be connected in series before installation. This enables the simultaneous installation of the plurality of light guide components 30, which avoids the complexity of assembling the light guide components 30 individually, thereby achieving quick installation.

In the embodiments, the connecting component 330 may be a plate-like structure, which envelops the peripheries of the plurality of light guide components 30, thereby connecting the multiple adjacent light guide components 30. The structure is simple and has a good integrity. In some other embodiments, the connecting component 330 may adopt other common structures, which is not limited herein.

In the embodiments, the connecting component 330 may be made of a light guide material for guiding the light emitted by the indicator lamp of the beauty device.

In some embodiments, the light guide components 30 and the connecting component 330 are integrally formed, for example, by means of injection molding. This allows for a stable connection of the plurality of light guide components 30, making them less likely to detach, and also reduces the number of parts, thereby lowering the processing cost and simplifying assembly.

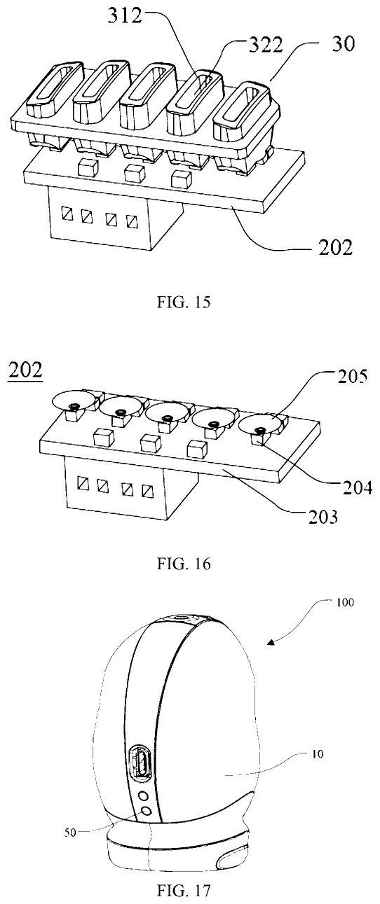

Referring to FIG. 14 and FIG. 15, in some embodiments, the beauty device 100 further includes a housing 10 and a light-emitting component 202 arranged on the housing 10.

In some embodiments, referring to FIG. 16, the light-emitting component 202 includes a light-emitting control circuit board 203 and an indicator lamp 204 electrically connected to the light-emitting control circuit board 203. The indicator lamp 204 is configured to emit light for a user to observe the working state of the beauty device 100. In some embodiments, the indicator lamp is a LED lamp bead 204.

In the embodiments, the LED lamp bead 204 may be a RGB lamp bead. The three primary colors in the RGB lamp bead are red, blue, and green, and through the combination of RGB, a variety of different colors can be formed, which in turn emit white light or other multicolored light for different color displays. In some other embodiments, the LED lamp bead 204 may be a white lamp bead, which is not limited herein.

In some embodiments, the LED lamp bead 204 may emit color-changing flashing light, and indicate different states by different light effects, which greatly enhances the user's experience. In some other embodiments, the LED lamp bead 204 may emit non-color-changing steady light, which is not limited herein.

It should be noted that the technical solution of the present disclosure is an improvement made to the light guide manner of the light guide component 30. The other parts and related functions of the beauty device 100 of the present disclosure are all known in related art, which is not detailed herein. Those skilled in the art should be aware of and proficient in these aspects.

The light guide component 30 is arranged corresponding to the light-emitting component 202. That is, the side wall 310 and the channel 320 are arranged corresponding to the light-emitting component 202. In this way, a part of the light emitted by the light-emitting component 202 enters from the end face 311 of the side wall 310 and exits from the other end face 312 of the side wall 310, and the other part of the light enters from the light inlet 321 of the channel 320 and exits from the light outlet 322. Thus, a part of the light emitted by the light-emitting component 202 is guided out by the side wall 310, and the other part of the light directly exits through the channel 320. This ensures the intensity of the light while guiding the light, thereby enhancing the indication effect of the beauty device 100 and facilitating a user to observe the working state of the beauty device 100.

The beauty device 100 provided in the present disclosure has better indication effects, solving the problem in the related art that the indicator lamp, when mounted on the side surface of the beauty device, does not provide sufficient light guidance intensity.

In the embodiments, the light-emitting component 202 includes a plurality of LED lamp beads 204. The on/off states and colors of the LED lamp beads 204 may correspond to different working states of the beauty device 100, such as the remaining battery power, working mode, or working duration, to facilitate a user to understand the working states of the beauty device 100.

Correspondingly, the number of the light guide components 30 is the same as the number of the LED lamp beads 204 of the light-emitting component 202, and the side wall 310 and the channel 320 of each light guide component 30 are arranged corresponding to one LED lamp bead 204 of the light-emitting component 202, such that the light emitted from each LED lamp bead 204 can exit through the side wall 310 and the channel 320 of the light guide component 30.

In some embodiments, referring to FIG. 16, the beauty device 100 further includes a condenser 205 arranged between the light-emitting component 202 and the light guide component 30. The condenser 205 is configured to converge the light, preventing it from diverging and ensuring that the light enters the light guide component 30 as much as possible. This improves the utilization rate of the light emission and thereby increasing the brightness of the emitted light.

In the embodiments, the condenser 205 may be a flared reflective cup, with openings at two ends. The diameter of the reflective cup gradually increases from the end close to the LED lamp bead 204 to the other end away from the LED lamp bead 204. That is, the small-diameter opening of the reflective cup is aligned with the LED lamp bead 204, and the large-diameter opening of the reflective cup faces away from the LED lamp bead 204. The light emitted from the LED lamp bead 204 enters from the small-diameter opening and exits from the large-diameter opening.

The light emitted by the LED lamp bead 204 is reflected by the reflective cup, and enters the light guide component 30, playing a role of converging the light. This helps to avoid the loss of light energy, thereby increasing the indication range and intensity of the LED lamp bead 204, making it easier for users to observe the working state of the beauty device 100.

In the embodiments, the reflective cup is arranged between the light-emitting component 202 and the light guide component 30. In this way, the reflective cup can enhance the intensity of the light entering the light guide component 30, and by way of the light-guiding and light-transmitting functions, the light guide component 30 help to enhance the light brightness of the beauty device 100.

In some embodiments, the small-diameter opening of the reflective cup is directly sleeved on the LED lamp bead 204 of the light-emitting component 202. In this way, all the light emitted by the LED lamp bead 204 can be converged by the reflective cup, thereby increasing the intensity of the light entering the light guide component 30.

In some embodiments, the plurality of light guide components 30 are mounted on a side surface of the housing 10, and adjacent light guide components 30 are connected by the connecting component 330.

Among the plurality of light guide components 30, the height of the light guide component 30 located in the middle of the connecting component 330 is higher than the heights of the light guide components 30 located on two sides of the connecting component 30. In other words, the upper surfaces of the plurality of light guide components 30 are in an arc shape.

In the embodiments, the shape of the light guide components 30 and the number of the LED lamp beads 204 may be specifically set according to actual needs. For example, when used only to indicate the on and off states of the beauty device 100, one LED lamp bead 204 is provided, and one light guide component 30 is provided correspondingly, as long as it ensures the transmission of the light from the LED lamp bead 204.

When used to display different working states of the beauty device 100, a plurality of LED lamp beads 204 and a plurality of light guide components 30 may be provided. In this case, the plurality of light guide components 30 are mounted on the side surface of the housing 10. The specific arrangement of the light guide components 30 requires corresponding structural designs, for example, the side surface of the housing 10 is arc-shaped. In order to adapt to the housing 10 and facilitate the display of the light-emitting conditions of the plurality of LED lamp beads 204, among the plurality of light guide components 30, the height of the light guide component 30 located in the middle of the connecting component 330 is higher than the heights of the light guide components 30 located on two sides of the connecting component 330. In other words, the plurality of light guide components 30 are arranged at intervals to form an arc shape, to adapt to the arc-shaped structure of the housing 10, thereby improving the light transmission. Even if the user's line of sight is not directly facing the LED lamp beads 204, they can still clearly observe the indication of the LED lamp beads 204.

The number of the light guide components 30 is same as the number of the LED lamp beads 204. The light emitted by each LED lamp bead 204 can be transmitted through its corresponding light guide component 30, which ensures that different indicator lights can be displayed, so as to display different working state information of the beauty device 100.

In some embodiments, a hole 206 is defined on the side surface of the housing 10, and a fixing structure 207 is arranged in the hole 206 for mounting the light guide component 30 and the light-emitting component 202. The fixing structure 207 may be a snap-fit structure, which allows a detachable installation of the light guide component 30 and the light-emitting component 202, making the assembly quick and convenient.

In some other embodiments, the fixing structure 207 may be a common fixing structure, which is not limited herein.

Further, the beauty device 100 further includes a light-transmitting component 208. The light-transmitting component 208 may be made of a transparent material and cover the surface of the light guide component 30. The light emitted by the LED lamp bead 204 exits through the light guide component 30 and the light-transmitting component 208, thereby further improving the uniformity of the light of the beauty device 100.

In some other embodiments, a hollowed-out window may be defined in the housing 10 corresponding to the light guide component 30, which allows the light emitted by the LED lamp bead 204 to be transmitted without obstruction to the greatest extent, facilitating the user to observe the working state of the beauty device 100.

In the embodiments, the plurality of light guide components 30 are arranged at intervals along the side surface of the housing 10, and correspondingly, the plurality of LED lamp beads 204 of the light-emitting component 202 are arranged at intervals along the side surface of the housing 10, to maximize the use of the space of the side surface of the housing 10, further enhancing the indication effect of the beauty device 100.

According to the embodiments, by providing the side wall 310 and the channel 320 defined by the side wall 310, the light emitted by the indicator lamp of the beauty device is divided into two parts that enter the light guide component 30: a part of the light enters from the end face 311 of the side wall 310 and exits from the other end face 312 of the side wall 310; the other part of the light enters from the light inlet 321 of the channel 320 and exits from the light outlet 322, such that a part of the light emitted by the indicator lamp of the beauty device is guided out through the side wall 310, and the other part of the light directly exits through the channel 320. This ensures the intensity of the light while guiding the light, enhancing the indication effect of the indicator lamp of the beauty device and making it easier for users to observe the working state of the beauty device.

The working principle of the beauty device is to use technologies such as micro-frequency current, radio frequency, ultrasound, and intense light to stimulate and act on the skin, to improve skin metabolism and contract muscles, thus achieving cosmetic effects. For example, a radio frequency beauty device uses a radio frequency energy for skin treatment. For another example, a microcurrent beauty device uses a microcurrent for skin treatment. This type of radio frequency or microcurrent beauty device mainly includes an electrode head that comes into contact with the skin and a control apparatus. The electrode head is controlled to generate a radio frequency signal or a microcurrent to stimulate the user's skin, achieving cosmetic effects. The radio frequency or microcurrent beauty device is generally provided with a charging electrode to charge the beauty device.

In the related art, the charging electrode of the beauty device is generally welded to the internal control circuit board by wires. After prolonged use, solder joints are prone to falling off, which will easily lead to poor contact, thereby losing their conductive function and affecting the normal operation of the beauty device. Moreover, due to the inability to stable clamping, the stability during welding and the accuracy of the welding cannot be guaranteed, which directly leads to low production efficiency and a low product qualification rate, increasing the difficulty of welding and adds to labor costs. Therefore, in order to solve the above technical problem, the present disclosure proposes at least the following embodiments.

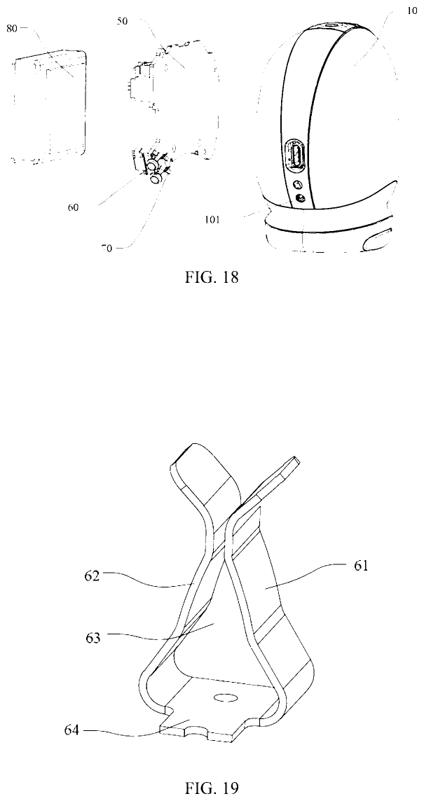

Referring to FIG. 17 and FIG. 18, in some embodiments, the beauty device 100 further includes a housing 10, a charging control circuit board 40 arranged in the housing 10, and a charging electrode 50 exposed outside the housing 10. The charging electrode 50 is configured to be electrically connected to an external power supply device.

In the embodiments, the charging electrode 50 is exposed outside the housing 10, which may include the situation that the charging electrode 50 protrudes from the housing 10, or the situation that the charging electrode 50 is flush with the surface of the housing 10, or the situation that the charging electrode 50 is recessed a certain distance from the surface of the housing 10. The specific arrangement is not limited herein, as long as the external power supply device can be electrically connected to the charging electrode 50 to charge the beauty device 100.

In the embodiments, a rechargeable battery 70 is arranged in the housing 10. The rechargeable battery 70 is electrically connected to the charging control circuit board 40, and the external power supply device is electrically connected to the charging electrode 50 to charge the rechargeable battery 70.

It should be understood that the technical solution of the present disclosure is an improvement made to the connection manner of the charging electrode 50 and the charging control circuit board 40. The other parts and related functions of the beauty device 100 of the present disclosure are all known in related art, which is not detailed herein. Those skilled in the art should be aware of and proficient in these aspects.

In some embodiments, the beauty device 100 further includes an elastic connecting component 60. An end of the elastic connecting component 60 is connected to the charging control circuit board 40, and the other end of the elastic connecting component 60 is connected to an end of the charging electrode 50. The elastic connecting component 60 conducts the electrical connection between the charging control circuit board 40 and the charging electrode 50. The other end of the charging electrode 50 is exposed outside the housing 10 for an electrical connection with the external power supply device. In this way, the elastic connecting component 60 connects the external power supply device, the charging electrode 50, and the charging control circuit board 40 to form a charging circuit, allowing the external power supply to charge the beauty device 100.

In the embodiments, the elastic connecting component 60 is provided. One end of the elastic connecting component 60 is connected to the charging control circuit board 40, the other end of the elastic connecting component 60 is connected to the charging electrode 50, and the elastic connecting component 60 conducts the electrical connection between the charging control circuit board 40 with the charging electrode 50, completing the assembly of the charging electrode 50. This kind of assembly is convenient and fast, which eliminates the welding process in the related art, greatly reducing the assembly difficulty and cost. Moreover, the connection is stable and reliable, ensuring a good electrical connection and a normal operation of the beauty device 100.

In the embodiments, a pair of charging electrodes 50 is provided, and a pair of elastic connecting components 60 is provided correspondingly. Each elastic connecting component 60 is electrically connected to one charging electrode 50 and the charging control circuit board 40 respectively. When the charging electrode 50 is electrically connected to the external power supply device, a charging circuit may be formed, and an electrical energy can be transmitted from the external power supply device to the beauty device 100, thereby charging the beauty device 100.

In some other embodiments, the number of the charging electrodes 50 may be other integers greater than 2. The number of the charging electrodes 50 is not limited herein.

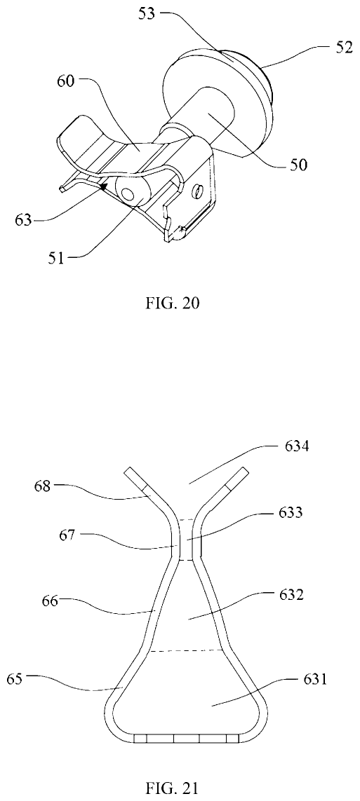

In some embodiments, referring to FIG. 19, the elastic connecting component 60 includes a first electrical conductor 61 and a second electrical conductor 62. At least one of the first electrical conductor 61 and the second electrical conductor 62 has elasticity. One end of the first electrical conductor 61 and/or one end of the second electrical conductor 62 is connected to the charging control circuit board 40, and the other end of the first electrical conductor 61 and the other end of the second electrical conductor 62 are close to each other to define an elastic clamp jaw 63. The charging electrode 50 is inserted into the elastic clamp jaw 63.

In an initial state, the diameter of the elastic clamp jaw 63 is smaller than the diameter of the charging electrode 50. In the process of snapping the charging electrode 50 into the elastic clamp jaw 63, the charging electrode 50 presses against at least one of the first electrical conductor 61 and the second electrical conductor 62 that has elasticity, realizing a pressing contact. This makes the contact between the charging electrode 50 and the elastic connecting component 60 more secure. Moreover, at least one of the first electrical conductor 61 and the second electrical conductor 62 is electrically connected to the charging electrode 50 to form an effective electrical connection. In the embodiments, the connection between the charging electrode 50 and the elastic connecting component 60 is highly reliable, preventing any accidental detachment or slipping of the charging electrode 50.

In the embodiments, at least one of the first electrical conductor 61 and the second electrical conductor 62 has elasticity. In other words, the first electrical conductor 61 or the second electrical conductor 62 has elasticity, or both the first electrical conductor 61 and the second electrical conductor 62 have elasticity, such that the elastic clamp jaw 63 with elasticity is able to be formed between the first electrical conductor 61 and the second electrical conductor 62, providing a stable elastic clamping and fixation for the charging electrode 50.

In some embodiments, both the first electrical conductor 61 and the second electrical conductor 62 have elasticity, to provide a more stable and reliable elastic clamping and fixation for the charging electrode 50.

In the embodiments, one end of the first electrical conductor 61 and/or one end of the second electrical conductor 62 is connected to the charging control circuit board 40. In other words, at least one of the first electrical conductor 61 and the second electrical conductor 62 is connected to the charging control circuit board 40, the electrical connection can be established between the charging control circuit board 40 and the charging electrode 50.

In some embodiments, both the first electrical conductor 61 and the second electrical conductor 62 are connected to the charging control circuit board 40, allowing for a stable connection between the elastic connecting component 60 and the charging control circuit board 40.

Referring to FIG. 20, in some embodiments, the charging electrode 50 includes a first end 51 and a second end 52 arranged along a length direction of the charging electrode 50 and facing the first end 51. The first end 51 is inserted into the elastic clamp jaw 63 for electrically connecting to the first electrical conductor 61 and/or the second electrical conductor 62, and the second end 52 is exposed outside the housing 10 for electrically connecting to the external power supply device.

In some embodiments, the charging electrode 50 is provided with an elongated shape, which may be directly assembled into the housing 10 and connected to the elastic connecting component 60 during assembly. This makes it convenient for assembly and compact in size, thus occupying a small space. In some other embodiments, the charging electrode 50 may be in other shapes, such as a curved shape. The shape of the charging electrode 50 is not limited herein, as long as one end of the charging electrode 50 is connected to the elastic connecting component 60, and the other end of the charging electrode 50 is exposed outside the housing 10.

In the embodiments, a brim portion 53 is provided at the second end 52 of the charging electrode 50, and the housing 10 is defined with a through hole 101 corresponding to the charging electrode 50. The brim portion 53 is mounted at the through hole 101 to fix the charging electrode 50. This prevents the charging electrode 50 from slipping out of the housing 10, and also facilitates the connection of the charging electrode 50 to the external power supply device.

In some embodiments, a radial dimension of the brim portion 53 is larger than a radial dimension of the through hole 101, preventing the charging electrode 50 from slipping out of the through hole 101 to the outside of the housing 10.

In some embodiments, the elastic connecting component 60 further includes a conductive component 64 connected to the charging control circuit board 40. The first electrical conductor 61 is fixedly connected to one end of the conductive component 64, and the second electrical conductor 62 is fixedly connected to the other end of the conductive component 64. The conductive component 64 conducts the electrical connection between the first electrical conductor 61 and/or the second electrical conductor 62 with the charging control circuit board 40.

In an actual assembling process, the conductive component 64 may be fixed on the charging control circuit board 40 by a screw. The first electrical conductor 61 is fixedly connected to one end of the conductive component 64, and the second electrical conductor 62 is fixedly connected to the other end of the conductive component 64.

In the embodiments, the conductive component 64 conducts the first electrical conductor 61 and/or the second electrical conductor 62 with the charging control circuit board 40. In other words, the conductive component 64 only needs to conduct at least one of the first electrical conductor 61 and the second electrical conductor 62 with the charging control circuit board 40.

In some embodiments, the conductive component 64, the first electrical conductor 61, and the second electrical conductor 62 are integrally formed, for example, by using a thin-walled metal plate through processes such as sheet metal stamping and stretching. Hence, the elastic connecting component 60 is simple to manufacture and has high production efficiency, further reducing production costs. Additionally, it enhances the integrity of the entire structure to make it less likely to separate, thus allowing the elastic connecting component 60 to have good electrical connection stability.

The first electrical conductor 61 is bent upward from one end of the conductive component 64, the second electrical conductor 62 is bent upward from the other end of the conductive component 64, and the two are close to each other to define the elastic clamp jaw 63. The elastic connecting component 60 according to the embodiments has a simple structure, which is convenient to manufacture and produce, and offers good electrical connection stability.

In some embodiments, the elastic connecting component 60 is a metal elastic plate, which has good conductive performance and elastic deformation performance. It realizes the elastic clamping fixation for the charging electrode 50 and realizes the electrical connection between the charging electrode 50 and the charging control circuit board 40.

Moreover, the metal elastic plate has large contact areas with the charging electrode 50 and the charging control circuit board 40, allowing for a high connection reliability. This ensures the beauty device 100 to be charged and operated normally.

In some embodiments, referring to FIG. 21, the elastic clamp jaw 63 includes a first region 631 and a second region 632 communicated with the first region 631. The size of the first region 631 is greater than the size of the second region 632. That is, the projection area of the first region 631 along a target direction is greater than the projection area of the second region 632 along the target direction. The target direction refers to the direction perpendicular to the opening direction of the elastic clamp jaw 63 and the deformation direction of the first electrical conductor 61 or the second electrical conductor 62.

The first electrical conductor 61 and the second electrical conductor 62 each includes a first section 65 and a second section 66. The first section 65 includes a first end and a second end facing the first end along the length direction of the first section 65. The second section 66 includes a first end and a second end facing the first end along the length direction of the second section 66. The first end of the first section 65 is connected with the conductive component 64, and the first end of the second section 66 is connected with the second end of the first section 65. The second ends of the two first sections 65 are close to each other to form the first region 631, and the second ends of the two second sections 66 are close to each other to form the second region 632. The charging electrode 50 is inserted in the second region 632.

The first sections 65 and the first region 631 defined by the first sections 65 are configured to provide a larger area for elastic deformation of the second sections 66. This facilitates the elastic deformation of the second sections 66, offering better elastic deformation performance. As such, when inserting the charging electrode 50, it can be pressed outward to form a stable electrical contact, and when removing the charging electrode 50, it can promptly return to its original shape.

In the embodiments, the two second sections 66 are arranged in a V-shaped structure to provide a good elastic clamping force. During assembly, the two second sections 66 elastically clamp the charging electrode 50, providing good stability and preventing the charging electrode 50 from slipping off easily. In some other embodiments, the two second sections 66 may be arranged in other shapes, such as a shape of a circle, a shape of an ellipse, which is not limited herein.

In some embodiments, the elastic clamp jaw 63 further includes a third region 633 communicated with the second region 632. The size of the third region 633 is less than the size of the second region 632. That is, the projection area of the third region 633 along a target direction is less than the projection area of the second region 632 along the target direction. The target direction refers to the direction perpendicular to the opening direction of the elastic clamp jaw 63 and the deformation direction of the first electrical conductor 61 or the second electrical conductor 62.

The first electrical conductor 61 and the second electrical conductor 62 each further includes a third section 67. A first end of the third section 67 is connected to the second end of the second section 66, and the two third sections 67 are close to each other to form the third region 633.

The third region 633 is in a tightened state relative to the second region 632, serving to tighten the first electrical conductor 61 and the second electrical conductor 62. This allows the two second sections 66 to come close to each other and generate an elastic clamping force, thereby more stably clamping the charging electrode 50.

In some embodiments, an inclination of each of the first section 65, the second section 66, and the third section 67 is gradually decreased, where the inclination refers to a degree of tilt relative to the opening direction of the elastic clamp jaw 63. In addition, the first section 65, the second section 66, and the third section 67 are each directed toward the inner side of the conductive component 64, respectively.

In the embodiments, the conductive component 64, the first section 65, the second section 66, and the third section 67 are connected to form a non-closed triangular shape, which is structurally stable and less likely to deviate, thus not affecting the fixation of the charging electrode 50. Moreover, it is compact and occupies minimal space.

In some embodiments, the elastic clamp jaw 63 further includes a fourth region 634.

The first electrical conductor 61 and the second electrical conductor 62 each further includes a fourth section 68. A first end of the fourth section 68 is connected to the second end of the third section 67, the two fourth sections 68 face away from each other from their first ends to their second ends to form the fourth region 634. The fourth region 634 is communicated to the third region 633.

During installation, the charging electrode 50 may be slid into the second region 632 from the fourth region 634 and the third region 633, and clamped by the two second sections 66; or, the charging electrode 50 may be slid into the second region 632 from the first region 631, and clamped by the two second sections 66.

In some embodiments, the fourth region 634 is in a flared shape. That is, the opening of the fourth region 634 gradually increases from the communication with the third region 633 toward the direction away from the third region 633, which facilitates the charging electrode 50 to slide into the third region 633 and the second region 632 from the fourth region 634. The fourth region 634 serves as a guide, improving the accuracy of inserting the charging electrode 50 and the convenience of installation.

In some other embodiments, the fourth region 634 may be in other shapes, which is not limited herein.

In some embodiments, the connection between the conductive component 64 and the first section 65, the connection between the first section 65 and the second section 66, the connection between the second section 66 and the third section 67, and/or the connection between the third section 67 and the fourth section 68 have curvature, that is, a smooth curvature transition is used to allow the elastic connecting component 60 to have good elastic deformation performance and prevent easy breakage.

In some embodiments, the other end of the elastic connecting component 60 is detachably connected to the charging electrode 50. The assembly and disassembly are realized by the elasticity of the connecting component 60. In this way, when the charging electrode 50 experiences poor contact or failure, a user may quickly detach the charging electrode 50 from the elastic connecting component 60 and replace it, ensuring continued use.

In summary, the embodiments provide the elastic connecting component 60. One end of the elastic connecting component 60 is connected to the charging control circuit board 40, the other end of the elastic connecting component 60 is connected to the charging electrode 50, and the elastic connecting component 60 conducts the electrical connection between the charging control circuit board 40 with the charging electrode 50, completing the assembly of the charging electrode 50. This kind of assembly is convenient and fast, which eliminates the welding process in the related art, greatly reducing the assembly difficulty and cost. Moreover, the connection is stable and reliable, ensuring a good electrical connection and a normal operation of the beauty device 100.

The above are only some embodiments of the present disclosure, and neither the words nor the drawings can limit the protection scope of the present disclosure. Any equivalent structural transformation made by using the contents of the specification and the drawings of the present disclosure under the overall concept of the present disclosure, or directly/indirectly applied in other related technical fields are included in the protection scope of the present disclosure.

Aspects of the beauty device described herein are further exemplified in the following numbered CLAUSES:

CLAUSE 1. A beauty device, comprising:
an isolation circuit, a first terminal of the isolation circuit being configured to access a test voltage or output a driving signal;
a working circuit, the working circuit being electrically connected to a second terminal of the isolation circuit, and configured to output the driving signal to the isolation circuit;
a housing; and
an electrode assembly arranged on the housing, the electrode assembly being at least partially exposed outside the housing;
wherein the electrode assembly comprises a first electrode group and a second electrode group arranged around the first electrode group;
the first electrode group comprises a first electrode and a second electrode that are spaced apart, and the first electrode and the second electrode have opposite polarities and form an electrode pair; and
a part of electrodes in the second electrode group has a polarity opposite to that of the first electrode and form an electrode pair with the first electrode, and the other part of the electrodes in the second electrode group has a polarity opposite to that of the second electrode and form an electrode pair with the second electrode.

CLAUSE 2. The beauty device according to CLAUSE 1, wherein both the first electrode and the second electrode are in a strip shape, and the first electrode is parallel to the second electrode.

CLAUSE 3. The beauty device according to CLAUSE 1, wherein both the first electrode and the second electrode have a bow-like shape, and the first electrode and the second electrode are arranged in mirror symmetry.

CLAUSE 4. The beauty device according to CLAUSE 1, wherein the first electrode is one of two first electrodes, the second electrode is arranged between the two first electrodes, and the second electrode and each first electrode of the two first electrodes form an electrode pair.

CLAUSE 5. The beauty device according to CLAUSE 4, wherein a length of the second electrode is greater than a length of the first electrode on either side of the second electrode.

CLAUSE 6. The beauty device according to any one CLAUSE 2 to CLAUSE 4, wherein the second electrode group comprises:
at least one third electrode;
wherein the at least one third electrode is arranged adjacent to the first electrode, and the third electrode and the first electrode have opposite polarities and form an electrode pair.

CLAUSE 7. The beauty device according to CLAUSE 6, wherein the second electrode group further comprises:
at least one fourth electrode;
wherein the at least one fourth electrode is arranged adjacent to the second electrode, and the fourth electrode and the second electrode have opposite polarities and form an electrode pair.

CLAUSE 8. The beauty device according to CLAUSE 7, wherein the at least one third electrode and the at least one fourth electrode have opposite polarities, and one third electrode of the at least one third electrode and one fourth electrode of the at least one fourth electrode that is adjacent to the one third electrode form an electrode pair.

CLAUSE 9. The beauty device according to CLAUSE 7, wherein the at least one third electrode and the at least one fourth electrode are spaced apart and around the first electrode and the second electrode.

CLAUSE 10. The beauty device according to CLAUSE 7, wherein the second electrode is arranged at a center of a circle enclosed by the at least one third electrode and the at least one fourth electrode, and the two first electrodes are symmetrically arranged on two sides of the second electrode.

CLAUSE 11. A beauty device, comprising:
an isolation circuit, a first terminal of the isolation circuit being configured to access a test voltage or output a driving signal;
a working circuit, the working circuit being electrically connected to a second terminal of the isolation circuit; and configured to output the driving signal to the isolation circuit;
a housing;
an indicator lamp arranged on the housing; and
a light guide component arranged corresponding to the indicator lamp;
wherein the light guide component comprises:
a side wall, an end face of the side wall being configured for light of the indicator lamp of the beauty device to enter, and the other end face of the side wall being configured for the light of the indicator lamp of the beauty device to exit; and
a channel defined by the side wall, an end of the channel being opened to define a light inlet for the light of the indicator lamp of the beauty device to enter, and the other end of the channel being opened to define a light outlet for the light of the indicator lamp of the beauty device to exit.

CLAUSE 12. The beauty device according to CLAUSE 11, wherein at least an inner end of the side wall close to where the light exits is provided with a reflective layer.

CLAUSE 13. The beauty device according to CLAUSE 12, wherein an outer surface of the reflective layer is an arc surface or an uneven surface.

CLAUSE 14. The beauty device according to CLAUSE 11, wherein the end face of the side wall for the light of the indicator lamp of the beauty device to enter is inclined.

CLAUSE 15. The beauty device according to CLAUSE 11, wherein the light guide component is one of a plurality of light guide components, the indicator lamp is one of a plurality of indicator lamps, and the plurality of light guide components are arranged at intervals and corresponding to the plurality of indicator lamps of the beauty device.

CLAUSE 16. The beauty device according to CLAUSE 15, wherein adjacent light guide components among the plurality of light guide components are spaced apart and connected by a connecting component.

CLAUSE 17. The beauty device according to CLAUSE 16, wherein the plurality of light guide components and the connecting component are integrally formed.

CLAUSE 18. The beauty device according to CLAUSE 11, further comprising: a condenser arranged between the indicator lamp and the light guide component.

CLAUSE 19. The beauty device according to CLAUSE 11, wherein the light guide component is one of a plurality of the light guide components, the plurality of light guide components are mounted on a side surface of the housing, and adjacent light guide components are spaced apart and connected by a connecting component; and among the plurality of light guide components, a height of the light guide component located at a middle of the connecting component is higher than a height of the light guide component located on either side of the connecting component.

CLAUSE 20. A beauty device, comprising:
an isolation circuit, a first terminal of the isolation circuit being configured to access a test voltage or output a driving signal;
a working circuit, the working circuit being electrically connected to a second terminal of the isolation circuit, and configured to output the driving signal to the isolation circuit;
a housing;
a charging control circuit board arranged in the housing;
a charging electrode exposed outside the housing; and
an elastic connecting component;
wherein an end of the elastic connecting component is connected with the charging control circuit board, the other end of the elastic connecting component is connected with the charging electrode, and the elastic connecting component conducts the charging control circuit board with the charging electrode.

CLAUSE 21. The beauty device according to CLAUSE 20, wherein the elastic connecting component comprises a first electrical conductor and a second electrical conductor;
wherein at least one of the first electrical conductor and the second electrical conductor is elastic, an end of the first electrical conductor and/or an end of the second electrical conductor is connected to the charging control circuit board, the other end of the first electrical conductor and the other end of the second electrical conductor are close to each other to define an elastic clamp jaw, and the charging electrode is inserted into the elastic clamp jaw.

CLAUSE 22. The beauty device according to CLAUSE 21, wherein the charging electrode comprises:
a first end; and
a second end arranged along a length direction of the charging electrode and facing the first end;
wherein the first end is inserted into the elastic clamp jaw, and the second end is exposed outside the housing.

CLAUSE 23. The beauty device according to CLAUSE 21, wherein
the elastic connecting component further comprises a conductive component connected to the charging control circuit board;
the first electrical conductor is fixedly connected to an end of the conductive component, and the second electrical conductor is fixedly connected to the other end of the conductive component; and
the conductive component conducts the first electrical conductor and/or the second electrical conductor with the charging control circuit board.

CLAUSE 24. The beauty device according to CLAUSE 23, wherein the elastic clamp jaw comprises a first region and a second region communicated with the first region;
the first electrical conductor and the second electrical conductor each comprises a first section and a second section connected to the first section;
both the first sections are connected to the conductive component, the two first sections are close to each other to define the first region, the two second sections are close to each other to define the second region, and the charging electrode is inserted in the second region.

CLAUSE 25. The beauty device according to CLAUSE 24, wherein the elastic clamp jaw further comprises a third region communicated with the second region;
the first electrical conductor and the second electrical conductor each further comprises a third section connected to the second section, and the two third sections are close to each other to form the third region.

CLAUSE 26. The beauty device according to CLAUSE 25, wherein an inclination of each of the first section, the second section and the third section is gradually decreased.

CLAUSE 27. The beauty device according to CLAUSE 25, wherein the elastic clamp jaw further comprises a fourth region communicated with the third region;
the first electrical conductor and the second electrical conductor each further comprises a fourth section connected to the third section, and the two fourth sections face away from each other to form the fourth region.

CLAUSE 28. The beauty device according to CLAUSE 27, wherein the fourth region is in a flared shape.

CLAUSE 29. The beauty device according to CLAUSE 23, wherein the conductive component, the first electrical conductor, and the second electrical conductor are integrally formed; and/or the elastic connecting component is a metal elastic plate.

CLAUSE 30. The beauty device according to any one of CLAUSE 20 to CLAUSE 29, wherein the other end of the elastic connecting component is detachably connected to the charging electrode.

What is claimed is:

1. A beauty device, comprising:
an isolation circuit, a first terminal of the isolation circuit being configured to access a test voltage or output a driving signal; and
a working circuit, the working circuit being electrically connected to a second terminal of the isolation circuit, and configured to output the driving signal to the isolation circuit.

2. The beauty device according to claim 1, wherein the working circuit comprises:
a control circuit; and
a driving circuit;
wherein the driving circuit is electrically connected to the control circuit and the isolation circuit, and the control circuit is configured to control the driving circuit to output the driving signal.

3. The beauty device according to claim 2, wherein
the beauty device further comprises:
a conductive assembly, the conductive assembly being configured to access the test voltage or output the driving signal; and
the isolation circuit comprises:
a transformer, a first terminal of the transformer being electrically connected to the conductive assembly, and a second terminal of the transformer being electrically connected to the driving circuit.

4. The beauty device according to claim 2, wherein
the beauty device further comprises:
a conductive assembly, the conductive assembly being configured to access the test voltage or output the driving signal;
the isolation circuit comprises:
a relay, a first terminal of the relay being electrically connected to the conductive assembly, a second terminal of the relay being electrically connected to the driving circuit, and a third terminal of the relay being electrically connected to the control circuit; and
the control circuit is configured to output a control signal to the third terminal of the relay to control a conduction between the first terminal and the second terminal of the relay, to allow the driving circuit to output the driving signal to the first terminal of the relay.

5. The beauty device according to claim 4, wherein
the beauty device further comprises a first resistor and a second resistor;
the first terminal of the relay comprises a first pin and a second pin, and the second terminal of the relay comprises a third pin and a fourth pin;
an end of the first resistor is electrically connected to the first pin, and the other end of the first resistor is electrically connected to the third pin;
an end of the second resistor is electrically connected to the second pin, and the other end of the second resistor is electrically connected to the fourth pin; and
the control circuit is configured to output the control signal to the third terminal of the relay to control a conduction between the first pin and the third pin, control a conduction between the second pin and the fourth pin, and allow the driving circuit to output the driving signal to the third pin and the fourth pin.

6. The beauty device according to claim 4, wherein
the isolation circuit further comprises a first switch circuit;
the third terminal of the relay comprises a fifth pin and a sixth pin;
the fifth pin is configured to access a power supply voltage, and the sixth pin is electrically connected to a first terminal of the first switch circuit, a second terminal of the first switch circuit is electrically connected to the control circuit, and a third terminal of the first switch circuit is grounded; and
the control circuit is configured to control a conduction between the first terminal and the third terminal of the first switch circuit, to control the conduction between the first terminal and the second terminal of the relay.

7. The beauty device according to claim 2, wherein
the beauty device further comprises:
a conductive assembly, the conductive assembly being configured to access the test voltage or output the driving signal;
the isolation circuit comprises:
an isolation optocoupler, a first terminal of the isolation optocoupler being electrically connected to the conductive assembly, a second terminal of the isolation optocoupler being electrically connected to the driving circuit, and a third terminal of the isolation optocoupler being electrically connected to the control circuit; and
the driving circuit is configured to output the driving signal to the first terminal of the isolation optocoupler.

8. The beauty device according to claim 7, wherein
the beauty device further comprises a second switch circuit, and the third terminal of the isolation optocoupler is electrically connected to the control circuit by the second switch circuit;
a first terminal of the second switch circuit is electrically connected to the third terminal of the isolation optocoupler, a second terminal of the second switch circuit is grounded, and a third terminal of the second switch circuit is electrically connected to the control circuit; and
the control circuit is configured to control a conduction between the first terminal and the second terminal of the second switch circuit, to control the isolation optocoupler to transmit the driving signal.

9. The beauty device according to claim 8, wherein
the isolation optocoupler comprises a first optocoupler and a second optocoupler;
wherein a first terminal of the first optocoupler is configured to access a power supply voltage, a second terminal of the first optocoupler is electrically connected to a first terminal of the second optocoupler, a third terminal of the first optocoupler is electrically connected to the driving circuit, and a fourth terminal of the first optocoupler is electrically connected to the conductive assembly;
a second terminal of the second optocoupler is electrically connected to the first terminal of the second switch circuit, a third terminal of the second optocoupler is electrically connected to the driving circuit, and a fourth terminal of the second optocoupler is electrically connected to the conductive assembly; and
the second terminal of the isolation circuit comprises the third terminal of the first optocoupler and the third terminal of the second optocoupler.

10. The beauty device according to claim 2, further comprising:
a power supply circuit, configured to output a power supply voltage to the isolation circuit and the working circuit; and
the control circuit is further configured to control the driving circuit to convert the power supply voltage into the driving signal for output.

11. The beauty device according to claim 1, further comprising:
a housing; and
an electrode assembly arranged on the housing, the electrode assembly being at least partially exposed outside the housing;
wherein the electrode assembly comprises a first electrode group and a second electrode group arranged around the first electrode group;
the first electrode group comprises a first electrode and a second electrode that are spaced apart, and the first electrode and the second electrode have opposite polarities and form an electrode pair; and
a part of electrodes in the second electrode group has a polarity opposite to that of the first electrode and form an electrode pair with the first electrode, and the other part of the electrodes in the second electrode group has a polarity opposite to that of the second electrode and form an electrode pair with the second electrode.

12. The beauty device according to claim 11, wherein
both the first electrode and the second electrode are in a strip shape, and
the first electrode is parallel to the second electrode.

13. The beauty device according to claim 11, wherein
both the first electrode and the second electrode have a bow-like shape, and
the first electrode and the second electrode are arranged in mirror symmetry.

14. The beauty device according to claim 11, wherein the first electrode is one of two first electrodes, the second electrode is arranged between the two first electrodes, and the second electrode and each first electrode of the two first electrodes form an electrode pair.

15. The beauty device according to claim 14, wherein a length of the second electrode is greater than a length of the first electrode on either side of the second electrode.

16. The beauty device according to claim 12, wherein the second electrode group comprises:
at least one third electrode;
wherein the at least one third electrode is arranged adjacent to the first electrode, the at least one third electrode and the first electrode have opposite polarities and form an electrode pair.

17. The beauty device according to claim 16, wherein the second electrode group further comprises:
at least one fourth electrode;
wherein the at least one fourth electrode is arranged adjacent to the second electrode, the at least one fourth electrode and the second electrode have opposite polarities and form an electrode pair.

18. The beauty device according to claim 17, wherein the at least one third electrode and the at least one fourth electrode have opposite polarities, and one third electrode of the at least one third electrode and one fourth electrode of the at least one fourth electrode that is adjacent to the one third electrode form an electrode pair.

19. The beauty device according to claim 17, wherein the at least one third electrode and the at least one fourth electrode are spaced apart and around the first electrode and the second electrode.

20. The beauty device according to claim 17, wherein the second electrode is arranged at a center of a circle enclosed by the at least one third electrode and the at least one fourth electrode, and two first electrodes are symmetrically arranged on two sides of the second electrode.

21-40. (canceled)
